(19) United States
(12) Patent Application Publication
(10) Pub. No.: US 2006/0270037 A1
(43) Pub. Date: Nov. 30, 2006

(54) COLLAGEN-COATED CARRIER AND METHOD FOR MANUFACTURING COLLAGEN-COATED CARRIER

(75) Inventors: Machiko KATO, Tokyo (JP); Ken SUGO, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/420,287

(22) Filed: May 25, 2006

(30) Foreign Application Priority Data
May 25, 2005 (JP) 2005-152778

(51) Int. Cl. C12N 5/08; A61F 2/28

(52) U.S. Cl. 435/402; 623/23.76; 623/23.51; 514/21; 427/2.27

(57) ABSTRACT

A collagen-coated carrier that has excellent cell adhesion properties and that allows excellent cell growth thereon is provided. Further, a method for manufacturing such a collagen-coated carrier efficiently and reliably is also provided. The collagen-coated carrier includes a base material (carrier) composed of a calcium phosphate-based compound and a coating layer provided so as to cover the surface of the base material. The coating layer is composed of collagen and a protein having a high affinity for the collagen. Such a coating layer covering the surface of the base material is formed by allowing the base material to firmly adsorb the collagen via the protein. The protein preferably has a collagen receptor. More preferably, the protein contains at least one of fibronectin and integrin as a main ingredient.

### BACKGROUND OF THE INVENTION

1. Field of the Invention

The present invention relates to a collagen-coated carrier and a method for manufacturing a collagen-coated carrier.

2. Description of the Prior Art

In recent years, cell culture technology is used in various industrial and research fields such as cell tissue engineering, safety tests of drugs, production of proteins for treatment and diagnosis purposes, and the like.

Currently, in order to culture a large number of anchorage-dependent cells efficiently, cell culture is carried out by not plane culture using a culture flask but by three-dimensional high-density culture (suspension culture) using carriers serving as scaffolds on which cells can grow.

In such three-dimensional high-density culture, various kinds of carriers such as those made of polystyrene, DEAE cellulose, or polyacrylamide and those composed of magnetic particles are used.

Meanwhile, in recent years, research in regenerative medicine has advanced rapidly. Regenerative medicine is a new medical therapy that is applicable to various tissues or organs within a body and that allows repair and regeneration of one's own tissue or organ by creating appropriate environment in a body with the use of a scaffold on which one's own cells can grow.

In order to culture cells efficiently, for example, carriers whose surfaces are coated with a calcium phosphate-based compound having high biocompatibility are used as scaffolds (see, for example, Japanese Patent Laid-open No, 2004-313007).

However, depending on the kind of cell to be cultured, there is a case where it is difficult to allow cells to reliably grow on such carriers due to their poor adhesion to the carriers. Under the circumstances, there is now demand for carriers to which even cells that are hard to adhere to such conventional carriers can reliably adhere.

In this regard, collagen is known as a material that promotes adhesion and growth of cells. Therefore, it can be considered that carriers whose surfaces are coated with collagen allow various cells to adhere thereto and grow thereon.

However, adsorption power of a calcium phosphate-based compound for collagen is very weak, thereby causing a problem that even when collagen is adsorbed to a calcium phosphate-based compound, the collagen easily comes off from the carriers

### SUMMARY OF THE INVENTION

It is therefore an object of the present invention to provide a collagen-coated carrier that has excellent cell adhesion properties and that allows excellent cell growth thereon and a method for manufacturing such a collagen-coated carrier efficiently and reliably.

In order to achieve the above object, the present invention is directed to a collagen-coated carrier, comprising a carrier having a surface, in which at least part of the surface of the carrier being composed of a calcium phosphate-based compound, wherein the part of the surface of the carrier is coated with collagen via a protein having a high affinity for the collagen.

According to the present invention, it is possible to obtain a collagen-coated carrier that has excellent cell adhesion properties and that allows excellent cell growth thereon.

In the collagen-coated carrier according to the present invention, it is preferred that the protein has a collagen receptor.

This allows the protein to selectively bind to the collagen so that the collagen is more firmly adsorbed to the carrier (base material).

In this case, the protein preferably contains at least one of fibronectin and integrin as a main ingredient.

This allows the collagen to be particularly firmly adsorbed to the carrier (base material) because fibronectin and integrin have a collagen receptor that selectively binds to collagen in their molecule, and has the property of firmly adsorbing a calcium phosphate-based compound.

In the collagen-coated carrier according to the present invention, it is also preferred that the collagen contains type I collagen as a main ingredient.

This is because type I collagen is present in large quantity in various kinds of tissues (organs) constituting a living body, and among various types of collagens, type I collagen has a high adsorptivity for various cells. In addition, type I collagen is relatively easily denatured, and has a high affinity for cells.

In the collagen-coated carrier according to the present invention, it is also preferred that the collagen is derived from a land animal.

This is because collagen derived from a land animal has a relatively high denaturation temperature, and is therefore relatively stable and hard to come off from the surface of the carrier at temperatures generally used for cell culture.

In the collagen-coated carrier according to the present invention, it is also preferred that at least part of the collagen is denatured.

This allows the collagen to have a higher affinity for cells so that a large number of cells are adsorbed to the collagen more firmly.

In the collagen-coated carrier according to the present invention, it is also preferred that the collagen can be dissolved in a solvent having a pH of 6.0 to 8.0 at a ratio of 100 μg or more per milliliter of the solvent.

This makes it possible to sufficiently dissolve the collagen in the solvent so that the collagen more reliably adheres to the surface of the carrier (base material) in the process of manufacturing a collagen-coated carrier. In addition, the use of such a solvent having a pH near neutrality for dissolving the collagen prevents the dissolution of the calcium phosphate-based compound.

In the collagen-coated carrier according to the present invention, it is also preferred that the carrier is obtained by coating the surface of a matrix with the calcium phosphate-based compound.

This makes it possible to obtain a base material having a more complicated shape while maintaining adhesion between the calcium phosphate-base compound (surface layer) and a coating layer covering the surface of the base material is composed of the collagen and the protein having a high affinity for the collagen.

In the collagen-coated carrier according to the present invention, it is also preferred that the carrier has a granular, pellet, block, or sheet shape.

This makes it possible to obtain a collagen-coated carrier having a granular, pellet, block, or sheet shape and to appropriately meet the demand for cell culture carriers or bone filling materials with a variety of shapes.

In the collagen-coated carrier according to the present invention, it is also preferred that the calcium phosphate-based compound contains at least one of tricalcium phosphate and hydroxyapatite as a main ingredient.

This is because tricalcium phosphate and hydroxyapatite have high biocompatibility, and therefore it is possible to obtain a carrier (base material) having a high affinity for a larger number of proteins.

The collagen-coated carrier according to the present invention is preferably used for cell culture.

In this case, cells to be cultured can grow more efficiently and reliably.

Also, the collagen-coated carrier according to the present invention is preferably used for filling a bone defect site.

In this case, the collagen-coated carrier and grown osteoblasts repair and regenerate the bone defect site faster.

Another aspect of the present invention is directed to a method for manufacturing a collagen-coated carrier, comprising the steps of:

preparing a carrier having a surface, in which at least part of the surface of the carrier being composed of a calcium phosphate-based compound; and

bringing the carrier into contact with collagen and a protein having a high affinity for the collagen to coat the part of the surface of the carrier with the collagen via the protein.

According to such a method, it is possible to manufacture a collagen-coated carrier efficiently and reliably.

Still another aspect of the present invention is directed to a method for manufacturing a collagen-coated carrier, comprising the steps of:

preparing a carrier having a surface, in which at least part of the surface of the carrier being composed of a calcium phosphate-based compound;

bringing the carrier into contact with a first treatment liquid containing a protein having a high affinity for collagen to allow the protein to adhere to the part of the surface of the carrier; and

bringing the carrier into contact with a second treatment liquid containing the collagen to coat the part of the surface of the carrier with the collagen via the protein.

According to such a method, it is possible to manufacture a collagen-coated carrier more efficiently and reliably.

In the manufacturing method according to still another aspect of the present invention, it is preferred that the protein concentration in the first treatment liquid is 0.1 to 100 μg/mL.

This allows the protein to be adsorbed to the carrier (base material) more efficiently and reliably.

In the manufacturing method according to still another aspect of the present invention, it is also preferred that the temperature of the first treatment liquid is 4 to 39° C.,

This allows the protein to be adsorbed to the carrier (base material) efficiently.

In the manufacturing method according to still another aspect of the present invention, it is also preferred that the time during which the carrier is kept in contact with the first treatment liquid is 10 minutes to 10 hours.

This allows the protein to be adsorbed to the carrier (base material) more efficiently.

In the manufacturing method according to still another aspect of the present invention, it is also preferred that the pH of the first treatment liquid is 6.0 to 8-0.

This makes it possible to properly prevent the protein and the calcium phosphate-based compound from being denatured and dissolved.

In the manufacturing method according to still another aspect of the present invention, it is also preferred that the collagen concentration in the second treatment liquid is 1 to 1,000 μg/mL.

This allows the collagen to be adsorbed to the protein more efficiently and reliably so that the coating layer is efficiently formed.

In the manufacturing method according to still another aspect of the present invention, it is also preferred that the temperature of the second treatment liquid is 4 to 39° C.

This allows the collagen to be reliably denatured and efficiently adsorbed to the protein.

In the manufacturing method according to still another aspect of the present invention, it is also preferred that the time during which the carrier is kept in contact with the second treatment liquid is 10 minutes to 10 hours.

This allows the collagen to be reliably denatured and more efficiently adsorbed to the protein.

In the manufacturing method according to still another aspect of the present invention, it is also preferred that the pH of the second treatment liquid is 6.0 to 8.0.

This makes it possible to properly prevent the aggregation/precipitation of the collagen in the second treatment liquid and to prevent the calcium phosphate-based compound from being dissolved.

### BRIEF DESCRIPTION OF THE DRAWINGS

FIG. 1 is a cross-sectional view of a first embodiment of a collagen-coated carrier according to the present invention;

FIG. 2 is a cross-sectional view of a second embodiment of the collagen-coated carrier according to the present invention;

FIG. 3 is an observation image of HUV-EC-C cells cultured using cell culture carriers obtained in Example 1;

FIG. 4 is an observation image of HUV-EC-C cells cultured using cell culture carriers obtained in Comparative Example 1;

FIG. 5 is an observation image of HUV-EC-C cells cultured using cell culture carriers obtained in Comparative Example 2; and

FIG. 6 is an observation image of HUV-CE-C cells cultured using cell culture carriers obtained in Comparative Example 3.

### BEST MODE FOR CARRYING OUT THE INVENTION

Hereinbelow, a collagen-coated carrier according to the present invention will be described in detail with reference to preferred embodiments shown in the accompanying drawings

### First Embodiment

First, a first embodiment of the collagen-coated carrier according to the present invention and a method for manufacturing the collagen-coated carrier will be described.

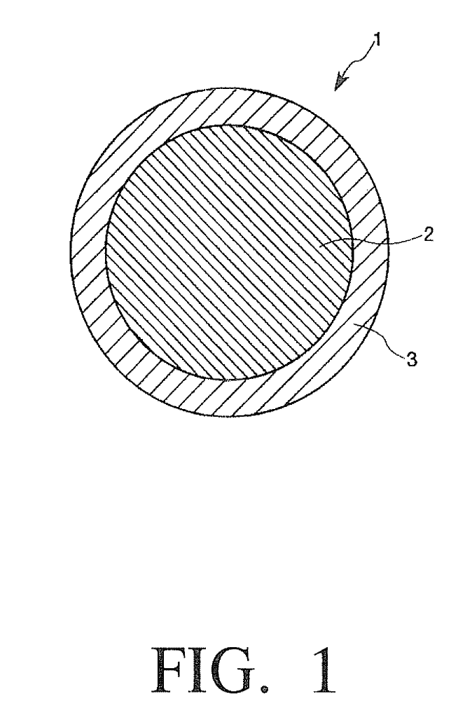

FIG. 1 is a cross-sectional view of the first embodiment of the collagen-coated carrier according to the present invention. As shown in FIG. 1, a collagen-coated carrier 1 comprises a base material (carrier) 2 composed of a calcium phosphate-based compound and a coating layer 3 provided so as to cover the surface of the base material 2.

Such a collagen-coated carrier 1 serves as a scaffold that allows cells to adhere to and grow on the surface thereof.

Examples of cells to be cultured using such collagen-coated carriers include, but are not limited to, various cells such as undifferentiated embryonic stem cells, undifferentiated mesenchymal stem cells, host cells for use in genetic recombination, and the like.

The base material 2 is formed so as to have a granular, pellet, block, or sheet shaper which makes it possible to obtain a collagen-coated carrier 1 having a granular, pellet, block, or sheet shape and to appropriately meet the demand for cell culture carriers or bone filling materials with a variety of shapes.

The collagen-coated carrier 1 having a granular, pellet, or sheet shape is preferably used as, for example, a cell culture carrier. By forming collagen-coated carriers 1 so as to have such a shape, it is possible to reduce variations in the shape of the individual collagen-coated carriers 1, thereby minimizing the influence of the shape of the cell culture carrier on cell culture.

Further, the collagen-coated carrier 1 having a granular or block shape is preferably used as, for example, a bone filling material. When a bone defect site is filled with such a bone filling material, bone tissues (osteoblasts) grow more efficiently on the bone filling material so that the bone defect site is repaired.

Particularly, the collagen-coated carriers 1 each having a granular shape can be reliably charged into a bone defect site even when the bone defect site has a complicated shape, so that the bone defect site is more reliably repaired.

On the other hand, the collagen-coated carrier 1 having a block shape is often shaped so as to fit into a bone defect sites. Therefore, in a case where a bone defect site is relatively large, the collagen-coated carrier 1 having a block shape is suitably used to more reliably repair the bone defect site.

The coating layer 3 covering the surface of the base material 2 is composed of collagen and a protein having a high affinity for the collagen. Such a coating layer 3 is formed by allowing the base material 2 to firmly adsorb the collagen via the protein.

Hereinbelow, the coating layer 3 will be described in detail. It is to be noted that in the following description, a protein having a high affinity for collagen is simply referred to as a “protein”.

Here, the term “collagen” refers to a fibrous scleroprotein contained in animal connective tissue. Such collagen has a high affinity for cells, and therefore can be used as a substrate that promotes adhesion (bonding) of cells to carriers and growth of the cells thereon.

Further, such collagen may be classified into five main types, types I to V according to their molecular structure. Collagen to be used in the present invention may be composed of any of these various types of collagens, but preferably contains type I collagen as a main ingredient. Type I collagen is present in large quantity in various tissues (organs) constituting a living body, and among various types of collagens, type I collagen has a high adsorptivity for various cells. In addition, as described later in more detail, type I collagen is relatively easily denatured and has a high affinity for cells. For these reasons, type I collagen is suitable for use in the present invention.

Examples of such collagen include those derived from land animals such as swine, bovine, sheep, and human; and those derived from fishes such as salmon, tuna, Atka mackerel, Alaska pollack, flatfish (including left-eyed flounder and right-eyed flounder), and shark.

Among them, collagens derived from land animals are preferably used in the present invention. Collagen derived from a land animal has a relatively high denaturation temperature, and is therefore relatively stable and hard to come off from the surface of the base material 2 at temperatures generally used for cell culture.

Further, it is preferred that at least part of the collagen to be used in the present invention is denatured. This allows the collagen to have a higher affinity for cells so that a large number of cells are adsorbed to the collagen more firmly.

Furthermore, it is preferred that the collagen to be used in the present invention can be dissolved in a solvent having a pH of 6.0 to 8.0 at a ratio of 100 μg or more per milliliter of the solvent, more preferably at a ratio of 1,000 μg or more per milliliter of the solvent. This makes it possible to sufficiently dissolve the collagen in the solvent so that the collagen more reliably adheres to the surface of the base material 2 in the process of manufacturing a collagen-coated carrier 1. In addition, the use of such a solvent having a pH near neutrality for dissolving the collagen prevents the dissolution of the calcium phosphate-based compound. Examples of such a solvent include various kinds of buffers and various kinds of water described later.

As described above, the collagen adheres to the base material 2 via the protein so as to cover the base material 2.

The protein to be used in the present invention is not particularly limited as long as it has a high affinity for the collagen, but it preferably has a collagen receptor. A collagen receptor is a site that exists in a protein and that specifically binds to collagen. Therefore, a protein having a collagen receptor can selectively bind to collagen, thereby allowing the base material 2 to more firmly adsorb the collagen.

Examples of such a protein include fibronectin, integrin, and laminin,

Further, it is preferred that the protein to be used in the present invention exhibits excellent adhesion to the calcium phosphate-based compound. This allows the base material 2 to firmly adsorb the protein.

Among the above-mentioned proteins, the protein to be used in the present invention preferably contains at least one of fibronectin and integrin as a main ingredient. This allows the base material 2 to more firmly adsorb the protein because fibronectin and integrin have a collagen receptor that selectively binds to collagen in their molecule, and have the property of firmly adsorbing a calcium phosphate-based compound.

Here, fibronectin is a glycoprotein that is contained also in the plasma membrane of cells. Fibronectin not only specifically binds to biological polymers such as collagen but also has the property of firmly adsorbing a calcium phosphate-based compound. Therefore, fibronectin exhibits a particularly high adsorptivity for the base material 2 and the collagen.

On the other hand, integrin is a receptor that specifically binds to collagen and the like, and has a particularly high adsorptivity for a calcium phosphate-based compound. Like fibronectin, integrin also exhibits a particularly high adsorptivity for the base material 2 and the collagen.

The amount of the collagen to be adsorbed to the base material (carrier) 2 is preferably in the range of about 1×10⁻⁶, to 1×10⁻³ g, more preferably in the range of about 1×10⁻⁵, to 1×10⁻⁴ gr per cm² of the surface area of the base material (carrier) 2,

If the amount of the collagen to be adsorbed to the base material (carrier) 2 is less than the above lower limit value, the effect of the collagen on cell adhesion is not sufficiently exhibited. On the other hand, if the amount of the collagen to be adsorbed to the base material (carrier) 2 exceeds the above upper limit value, there is a fear that excessive collagen will come off from the base material (carrier) 2. Even if the amount of the coating layer 3 is increased to exceed the above upper limit value, it cannot be expected that the number of cells to be adhered to the coating layer 3 is further increased.

It is to be noted that the coating layer 3 preferably covers almost all the surface of the base material (carrier) 2 from the viewpoint of allowing a larger number of cells to adhere thereto and grow thereon. However, the present invention is not limited to one having such a structure. Specifically, the coating layer 3 may cover only part of the surface of the base material 2. In this case, the surface of the base material 2 may be partially exposed.

The base material (carrier) 2 constitutes the framework of the collagen-coated carrier 1, and as described above, it is mainly composed of a calcium phosphate-based compound.

Examples of such a calcium phosphate-based compound include tricalcium phosphate, hydroxyapatite, and halogenated apatites such as fluoroapatite. Particularly, a calcium phosphate-based compound to be used in the present invention preferably contains at least one of tricalcium phosphate and hydroxyapatite as a main ingredient.

Tricalcium phosphate is close to the inorganic component of human bone in its composition and structure, and is highly biocompatible. Therefore, tricalcium phosphate has a high affinity for the protein described above, and can adsorb the coating layer 3 more firmly.

Hydroxyapatite has a unique crystalline structure resulting from its apatite structure, and therefore it has an especially high biocompatibility among various calcium phosphate-based compounds and has a high affinity for various proteins.

Hereinbelow, a method for manufacturing such a collagen-coated carrier 1 (that is, a collagen-coated carrier manufacturing method according to the present invention) will be described.

The collagen-coated carrier 1 can be manufactured by bringing the base material (carrier) 2 into contact with the collagen and the protein. According to such a method, it is possible to efficiently and reliably manufacture the collagen-coated carrier 1 in which the base material 2 is covered with the coating layer 3 formed by allowing the collagen to adhere to the surface of the base material 2 via the protein.

Specific examples of a method for bringing the base material (carrier) 2 into contact with the collagen and the protein include a method comprising bringing the base material 2 into contact with a treatment liquid containing both the collagen and the protein; and a method comprising bringing the base material 2 into contact with a treatment liquid containing the protein (hereinafter, also simply referred to as a “first treatment liquid”) and bringing the base material 2, which has been brought into contact with the first treatment liquid, into contact with a treatment liquid containing the collagen (hereinafter, also simply referred to as a “second treatment liquid”). Among these methods, the latter method is preferably employed. By employing the latter method, it is possible to more efficiently and reliably form a coating layer 3 firmly adsorbed to the base material 2.

Hereinbelow, the latter method will be described in more detail.

(1) First, a base material (carrier) 2 is brought into contact with a first treatment liquid containing a protein having a high affinity for collagen (hereinafter, simply referred to as a “protein”) to allow the protein to adhere to the surface of the base material 2.

Examples of a method for bringing the base material 2 into contact with the first treatment liquid include a method comprising immersing the base material 2 in the first treatment liquid (hereinafter, simply referred to as a “immersion method”), a method comprising applying the first treatment liquid onto the base material 2 (hereinafter, simply referred to as a “application method”), and a method comprising spraying a fine mist of the first treatment liquid to the base material 2 (hereinafter, simply referred to as a “spraying method”). Among these methods, the immersion method is preferably employed. According to the immersion method, it is possible to evenly bring a large number of base materials 2 into contact with the first treatment liquid.

Further, in a case where the immersion method is employed, the first treatment liquid is preferably stirred or shaken while a large number of the base materials 2 are being immersed therein. By doing so, it is possible to treat the base materials 2 evenly and speedily.

The first treatment liquid is prepared by dissolving the protein in a solvent (dispersion medium). Examples of such a solvent include various kinds of buffers (liquid containing a buffering agent) such as triethanolamine hydrochloride-sodium hydroxide buffer, veronal (sodium 5,5-diethyl barbiturate)-hydrochloric acid buffer, tris-hydrochloric acid buffer, glycylglycin-sodium hydroxide buffer, 2-amino-2-methyl-1,3-propanediol-hydrochloric acid buffer, diethanolamine-hydrochloric acid buffer, boric acid buffer, sodium borate-hydrochloric acid buffer, glycin-sodium hydroxide buffer, sodium carbonate-sodium bicarbonate buffer, sodium borate-sodium hydroxide buffer, sodium bicarbonate-sodium hydroxide buffer, phosphoric acid buffer, potassium phosphate-disodium phosphate buffer, disodium phosphate-sodium hydroxide buffer, potassium chloride-sodium hydroxide buffer, Briton-Robinson buffer, and GTA buffer; and various kinds of water such as pure water, ultrapure water, and ion-exchange water. Among these solvents, phosphoric acid buffer (PBS) is preferably used.

The protein concentration in the first treatment liquid is preferably in the range of about 0.1 to 100 μg/mL, more preferably in the range of about 0.5 to 50 μg/mL. By setting the protein concentration to a value within the above range, it is possible to allow the protein to be more efficiently and reliably adsorbed to the base material 2. However, even if the protein is added to the first treatment liquid so that the protein concentration in the first treatment liquid exceeds the above upper limit value, it cannot be expected that the efficiency of protein adsorption is further enhanced.

The temperature of the first treatment liquid to be brought into contact with the base material 2 is preferably in the range of about 4 to 39° C., more preferably in the range of about 15 to 38° C. By setting the temperature of the first treatment liquid to a value within the above range, it is possible to allow the protein to be efficiently adsorbed to the base material 2. If the temperature of the first treatment liquid exceeds the above upper limit value, there is a fear that the protein will be denatured.

The time during which the base material 2 is kept in contact with the first treatment liquid is preferably in the range of about 10 minutes to 10 hours, more preferably in the range of about 20 minutes to 1 hour. By setting the time during which the base material 2 is kept in contact with the first treatment liquid to a value within the above range, it is possible to allow the protein to be more efficiently adsorbed to the base material 2. However, even if the time during which the base material 2 is kept in contact with the first treatment liquid is set so as to exceed the above upper limit value, it cannot be expected that the efficiency of protein adsorption to the base material 2 is further enhanced.

The pH of the first treatment liquid is preferably in the range of about 6.0 to 8.0, more preferably in the range of bout 6.8 to 7.4. By setting the pH of the first treatment liquid to a value within the above range, it is possible to properly prevent the protein and the calcium phosphate-based compound from being denatured and dissolved.

(2) Next, the base material 2 to which the protein has been adsorbed is brought into contact with a second treatment liquid containing collagen so that the collagen adheres to the base material 2 via the protein. In this way, a coating layer 3 is formed.

The base material 2 can be brought into contact with the second treatment liquid in the same manner as in the step (1) described above.

The second treatment liquid is prepared by dissolving collagen in a solvent (dispersion medium). As such a solvent, the same one as used in the step (1) described above can be used.

The collagen concentration in the second treatment liquid is preferably in the range of about 1 to 1,000 μg/mL, more preferably in the range of about 5 to 500 μg/mL. By setting the collagen concentration in the second treatment liquid to a value within the above range, it is possible to allow the collagen to be adsorbed to the protein more efficiently and reliably so that a coating layer 3 is efficiently formed. However, even if the collagen is added to the second treatment liquid so that the collagen concentration in the second treatment liquid exceeds the above upper limit value, it cannot be expected that the efficiency of forming a coating layer 3 is further enhanced.

The temperature of the second treatment liquid to be brought into contact with the base material 2 is preferably in the range of about 4 to 39° C., more preferably in the range of about 15 to 38° C. By setting the temperature of the second treatment liquid to a value within the above range, it is possible to reliably denature the collagen and to allow the collagen to be efficiently adsorbed to the protein.

The time during which the base material 2 is kept in contact with the second treatment liquid is preferably in the range of about 10 minutes to 10 hours, more preferably in the range of about 20 minutes to 1 hour. By setting the time during which the base material 2 is kept in contact with the second treatment liquid to a value within the above range, it is possible to reliably denature the collagen and to allow the collagen to be more efficiently adsorbed to the protein. However, even if the time during which the base material 2 is kept in contact with the second treatment liquid is set to exceed the above upper limit value, it cannot be expected that the efficiency of collagen adsorption to the protein is further enhanced.

The pH of the second treatment liquid is preferably in the range of about 6.0 to 8.0, more preferably in the range of about 6.8 to 7.4. By setting the pH of the second treatment liquid to a value within the above range, it is possible to properly prevent the aggregation/precipitation of the collagen in the second treatment liquid and to prevent the calcium phosphate-based compound from being dissolved.

It is to be noted that in the collagen-coated carrier manufacturing method described above, the collagen may be subjected to treatment for denaturation before or after being adsorbed to the base material 2.

The treatment for denaturing the collagen can be carried out by, for example, a method comprising maintaining the second treatment liquid containing collagen at a predetermined temperature.

In this method for denaturing the collagen, the predetermined temperature is preferably in the range of about 1 to 80° C., more preferably in the range of about 25 to 40° C. By setting the predetermined temperature to a value within the above range, it is possible to more reliably denature the collagen.

Further, in the method for denaturing the collagen, the time during which the second treatment liquid is maintained at a predetermined temperature is not particularly limited, but is preferably in the range of about 10 minutes to 10 hours, more preferably in the range of about 30 minutes to 90 minutes. By setting the time to a value within the above range, it is possible to more reliably denature the collagen. However, even if the time is set to exceed the above upper limit value, it cannot be expected that denaturation of the collagen further proceeds.

Furthermore, in the method for denaturing the collagen, the pH of the second treatment liquid is preferably in the range of about 60 to 8.0, more preferably in the range of about 6.8 to 7.4. By setting the pH of the second treatment liquid to a value within the above range, it is possible to properly prevent the aggregation/precipitation of the collagen in the second treatment liquid.

It is to be noted that the coating layer 3 preferably covers the entire surface of the base material 2 from the viewpoint of allowing a larger number of cells to adhere to the surface of the collagen-coated carrier 1 and grow thereon. However, the coating layer 3 may cover only part of the surface of the base material 2.

### Second Embodiment

Hereinbelow, a second embodiment of the collagen-coated carrier according to the present invention will be described.

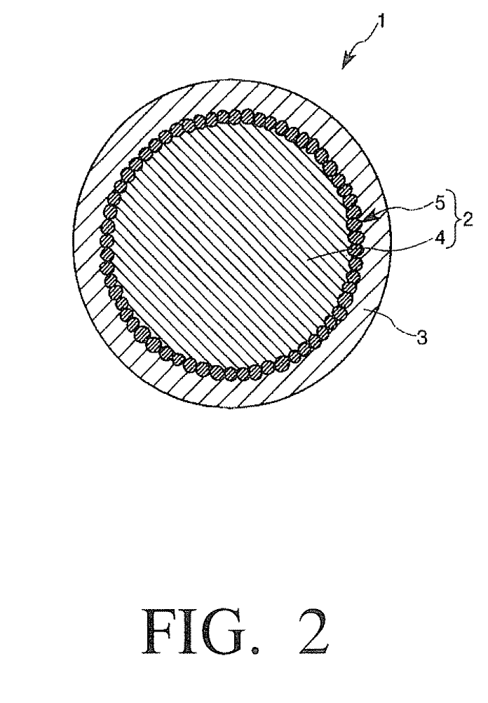

FIG. 2 is a cross-sectional view of the second embodiment of the collagen-coated carrier according to the present invention. In the following, the collagen-coated carrier of the second embodiment will be explained by focusing the difference between the first and second embodiments, and explanation with reference to the overlapping points is omitted.

A collagen-coated carrier 1 of the second embodiment shown in FIG. 2 is the same as the first embodiment except that a base material 2 comprises a matrix 4 and a surface layer 5. The surface layer 5 is composed of a calcium phosphate-based compound and covers the surface of the matrix 4.

According to such a structure of the base material 2, it is possible to obtain a base material 2 having a more complicated shape while maintaining adhesion between the calcium phosphate-based compound (surface layer 5) and the coating layer 3.

As a constituent material of the matrix 4, various ceramic materials and various resin materials can be mentioned. Examples of ceramic materials include, in addition to the above-mentioned calcium phosphate-based compounds, aluminum oxide, zirconium phosphate, silicate glass, and carbon-based compounds.

Examples of resin materials include various thermosetting resins and various thermoplastic resins. Specific examples of thermoplastic resins include polyamide, polyethylene, polypropylene, polystyrene, polyimide, acrylic resins, and thermoplastic polyurethane. Specific examples of thermosetting resins include epoxy resins, phenol resins, melamine resins, urea resins, unsaturated polyesters, alkyd resins, thermosetting polyurethane, and ebonite. These resins can be used singly or in combination of two or more of them.

The above-mentioned various materials themselves are often used as biomaterials due to their high level of safety for a living body. For this reason, these materials are suitable for use as constituent materials of the matrix 4.

The matrix 4 may be dense but is preferably porous. A porous matrix 4 allows the surface layer 5 (which will be described later) to easily penetrate pores in the surface of the matrix 4 so that an anchor effect is obtained. As a result, adhesive strength between the matrix 4 and the surface layer 5 is increased, thereby enabling a more stable base material 2 to be obtained.

As a calcium phosphate-based compound constituting the surface layer 5, the above-mentioned various calcium phosphate-based compounds can be used.

Further, the average thickness of the surface layer 5 is not particularly limited, but is preferably in the range of about 0.1 to 5 μm, more preferably in the range of about 0.5 to 2 μm.

The surface of the matrix 4 can be covered with the surface layer 5 composed of a calcium phosphate-based compound by, for example, a method comprising colliding particles each composed of a calcium phosphate-based compound with the surface of the matrix 4. According to such a method, it is possible to form a surface layer 5 easily and reliably.

It is to be noted that the base material 2 preferably has a structure in which the entire surface of the matrix 4 is covered with a calcium phosphate-based compound, from the viewpoint of allowing a larger number of cells to adhere to the surface of the collagen-coated carrier 1 and grow thereon. However, the present invention is not limited to one having such a structure. Specifically, the base material 2 may have a structure in which the surface of the matrix 4 is partially covered with a calcium phosphate-based compound. In this case, the rest of the surface of the matrix 4 may be exposed.

Both of the collagen-coated carrier 1 of the first embodiment and the collagen-coated carrier 1 of the second embodiment can be suitably used for, for example, cell culture technology used in various fields such as cell tissue engineering, safety tests of drugs, and production of proteins for treatment and diagnosis purposes. By using the collagen-coated carrier 1 of the present invention for such cell culture technology, it is possible to more efficiently and reliably grow cells to be cultured.

Particularly, in a case where cell culture is carried out by three-dimensional high-density culture (suspension culture) among various cell culture techniques, the collagen-coated carrier 7 of the second embodiment is preferably used.

In this case, the collagen-coated carrier 1 preferably has a granular (substantially spherical) shape. The collagen-coated carriers 1 each having a granular shape can be more uniformly suspended in a culture medium so that the collagen-coated carriers 1 have more opportunities to come into contact with cells, thereby enabling the cells to more efficiently adhere to the collagen-coated carriers 1.

At this time, the size of the collagen-coated carrier 1 is not particularly limited. However, when the maximum length of a cell (cell to be adhered to the collagen-coated carrier 1) is defined as L1 (μm) and the size of the collagen-coated carrier 1 is defined as L2 (μm), L2/L1 is preferably in the range of about 2 to 100, more preferably in the range of about 5 to

More specifically, L2 is preferably in the range of about 10 to 2,000 μm, more preferably in the range of about 50 to 1,000 μm, even more preferably in the range of about 100 to 300 μm.

By setting the size of the collagen-coated carrier 1 to a value within the above range, it is possible to sufficiently increase the surface area of the collagen-coated carrier 1 with respect to the size of the cell, thereby allowing the cells to adhere to and grow on the collagen-coated carrier 1 more easily.

Further, in three-dimensional high-density culture, it is necessary to more uniformly suspend the collagen-coated carriers 1 in a culture medium. Therefore, the density of the collagen-coated carrier 1 is preferably close to that of water. More specifically, the density of the collagen-coated carrier 1 is preferably in the range of about 101 to 1.5 g/cm³, more preferably in the range of about 102 to 1.2 g/cm³. By setting the density of the collagen-coated carrier 1 to a value within the above ranger it is possible to more uniformly suspend the collagen-coated carriers 1 in a culture medium, thereby allowing cells to more efficiently adhere to the collagen-coated carriers 1. The density of the collagen-coated carrier 1 of the second embodiment can be adjusted by appropriately setting, for example, the constituent material and form (e.g., porous or hollow structure) of the matrix 4. From such a viewpoint, the collagen-coated carrier 1 of the second embodiment can be suitably used in three-dimensional high-density culture.

The shape, size (e.g., average particle size), physical properties (e.g., density) etc of the collagen-coated carrier 1 can be adjusted by appropriately setting the shape, size, physical properties, etc. of the base material 2.

On the other hand, the collagen-coated carrier 1 can also be used as a scaffold (bone filling material) to be charged into, for example, a bone defect site to allow bone cells (osteoblasts) to grow thereon. In this case, the collagen-coated carrier(s) 1 and grown osteoblasts repair and regenerate the bone defect site faster.

Specific examples of the collagen-coated carriers 1 to be charged into a bone defect site include, in addition to those each having a granular shape, those each having a block shape, such as cranial plates, vertebral arch spacers, cervical vertebral spacers, artificial auditory ossicles, and artificial dental roots.

In a case where the collagen-coated carrier 1 is used as a bone filling material, the collage-coated carrier 1 of the first embodiment is preferred.

The collagen-coated carrier 1 can also be used as, for example, a stationary phase material for chromatography.

Although the collagen-coated carrier and the method for manufacturing a collagen-coated carrier according to the present invention have been described above, the present invention is not limited thereto.

For example, the method for manufacturing a collagen-coated carrier may further comprise one or two or more additional steps for any purpose, if necessary.

### EXAMPLES

Hereinbelow, actual examples of the present invention will be described.

1 Manufacture of Cell Culture Carrier

In each of the following Examples and Comparative Examples, ten collagen-coated carriers were manufactured in the following manner.

### Example 1

<1-1> First, fibronectin (which is a protein having a high affinity for collagen) was added to PBS (solvent) so that the fibronectin concentration in the PBS was 5 μg/mL, and they were mixed to prepare a fibronectin solution (first treatment liquid). The pH of the fibronectin solution was adjusted to 7.4.

<1-2> Next, one pellet (base material) of hydroxyapatite having a diameter of 5 mm and a thickness of 2 mm was immersed in 1.5 mL of the fibronectin solution having a temperature of 37° C., and was left standing for 30 minutes in the fibronectin solution being stirred. Thereafter, the pellet was taken out from the fibronectin solution, and was then washed with PBS.

<1-3> Next, type I collagen derived from swine was added to PBS (solvent) so that the type I collagen concentration in the PBS was 100 μg/mL, and they were mixed to prepare a collagen solution (second treatment liquid). The pH of the collagen solution was adjusted to 7.4.

<1-4> Next, the pellet obtained in the step <1-2> was immersed in 15 mL of the collagen solution adjusted to 37° C. for sufficiently denaturing the collagen, and was left standing for 30 minutes in the collagen solution being stirred.

Thereafter, the pellet was taken out from the collagen solution, and was then washed with PBS to obtain a collagen-coated carrier (hereinafter, also referred to as a “cell culture carrier”),

The above steps <1-1> to <1-4> were repeatedly carried out to finally obtain 10 collagen-coated carriers

### Example 2

Ten collagen-coated carriers were obtained in the same manner as in the Example 1 except that the constituent material of the base material was changed from hydroxyapatite to tricalcium phosphate

### Example 3

Ten collagen-coated carriers were obtained in the same manner as in the Example 1 except that the constituent material of the base material was changed from hydroxyapatite to a mixture comprising 50 wt % of hydroxyapatite and 50 wt % of tricalcium phosphate.

### Example 4

Ten collagen-coated carriers were obtained in the same manner as in the Example 1 except that the base material was changed from one composed of hydroxyapatite to one obtained by coating a matrix composed of a polystyrene resin with a surface layer composed of hydroxyapatite. The average thickness of the surface layer was 0.7 μm.

### Example 5

Ten collagen-coated carriers were obtained in the same manner as in the Example 1 except that the protein was changed from fibronectin to integrin.

### Example 6

Ten collagen-coated carriers were obtained in the same manner as in the Example 1 except that the protein was changed from fibronectin to a mixture comprising 50 parts by weight of fibronectin and 50 parts by weight of integrin.

### Example 7

Ten collagen-coated carriers were obtained in the same manner as in the Example 1 except that the collagen was changed from type I collagen to type II collagen.

### Example 8

Ten collagen-coated carriers were obtained in the same manner as in the Example 1 except that the collagen was changed from type I collagen derived from swine to type I collagen derived from salmon.

### Example 9

Ten collagen-coated carriers were obtained in the same manner as in the Example 1 except that the concentration of fibronectin in the fibronectin solution obtained in the step <1-1> was changed to 0.1 μg/mL.

### Example 10

Ten collagen-coated carriers were obtained in the same manner as in the Example 1 except that the concentration of fibronectin in the fibronectin solution obtained in the step <1-1> was changed to 100 μg/mL.

### Example 11

Ten collagen-coated carriers were obtained in the same manner as in the Example 1 except that the concentration of type I collagen in the collagen solution obtained in the step <1-3> was changed to 1 μg/mL.

### Example 12

Ten collagen-coated carriers were obtained in the same manner as in the Example 1 except that the concentration of type I collagen in the collagen solution obtained in the step <1-3> was changed to 1,000 μg/mL.

### Example 13

<13-1> First, fibronectin (which is a protein having a high affinity for collagen) and type I collagen derived from swine were added to PBS (solvent) so that the fibronectin concentration and the type I collagen concentration in the PBS were 5 μg/mL and 100 μg/mL, respectively, and then they were mixed to prepare a mixed solution (treatment liquid). The pH of the mixed solution was adjusted to 7.4.

<13-2> Next, one pellet (base material) of hydroxyapatite having a diameter of 5 mm and a thickness of 2 mm was immersed in 1.5 mL of the mixed solution having a temperature of 37° C., and was left standing for 30 minutes in the mixed solution being stirred.

Thereafter, the pellet was taken out from the mixed solution, and was then washed with PBS. The steps <13-1> and <13-2> were repeatedly carried out to finally obtain 10 collagen-coated carriers

### Comparative Example 1

Ten cell culture carriers were obtained in the same manner as in the Example 1 except that the steps <1-1> and <1-2> were omitted and that an untreated pellet of hydroxyapatite was used in the step <1-4>

### Comparative Example 2

Ten cell culture carriers were obtained in the same manner as in the Example 1 except that the steps <1-3> and <1-4> were omitted.

### Comparative Example 3

Ten untreated pellets of hydroxyapatite were prepared, and they were directly used as cell culture carriers.

2 Evaluation

2.1 Evaluation of Collagen Adsorption Power

Five of the ten cell culture carriers obtained in each of the Examples 1 to 13 and the Comparative Example 1 were washed with PBS, and the PBS was recovered to evaluate the collagen concentration in the PBS by electrophoresis (DDS-PAGE).

As a result, in all the cases of the Examples 1 to 13, the collagen concentration in the PBS with which the cell culture carriers had been washed was lower than that of the case of the Comparative Example 1.

From the result, it can be considered that the collagen of the cell culture carriers of each of the Examples 1 to 13 was firmly adsorbed to their base materials via the protein so that the collagen did not easily come off from the base materials even when the cell culture carriers were washed with PBS.

On the other hand, as described above, the collagen concentration in PES with which the cell culture carriers of the Comparative Example 1 had been washed was high. From the result, it can be considered that the collagen of the cell culture carriers of the Comparative Example 1 was weakly adsorbed to their base materials so that the collagen came off from the base materials due to washing with PBS.

2.2 Evaluation of Cell Growth

Cell culture was carried out using the remaining five cell culture carriers obtained in each of the Examples 1 to 13 and the Comparative Examples 1 to 3 in the following manner.

<2-1> First, normal human umbilical vein endotlhelial cells (hereinafter, simply referred to as “HUV-EC-C cells”) were inoculated at a ratio of 1.35×10⁵ cells per one cell culture carrier.

<2-2> Next, the HUV-EC-C cells inoculated in the step <2-1> were cultured in MCDB131 medium containing 10 wt % fetal calf serum (FCS) for 7 days. The temperature of the culture medium was 37° C. during cell culture.

Thereafter, the HUV-EC-C cells cultured using the cell culture carriers of each of the Examples 1 to 13 and the Comparative Examples 1 to 3 were stained with crystal violet, and were then observed with a microscope.

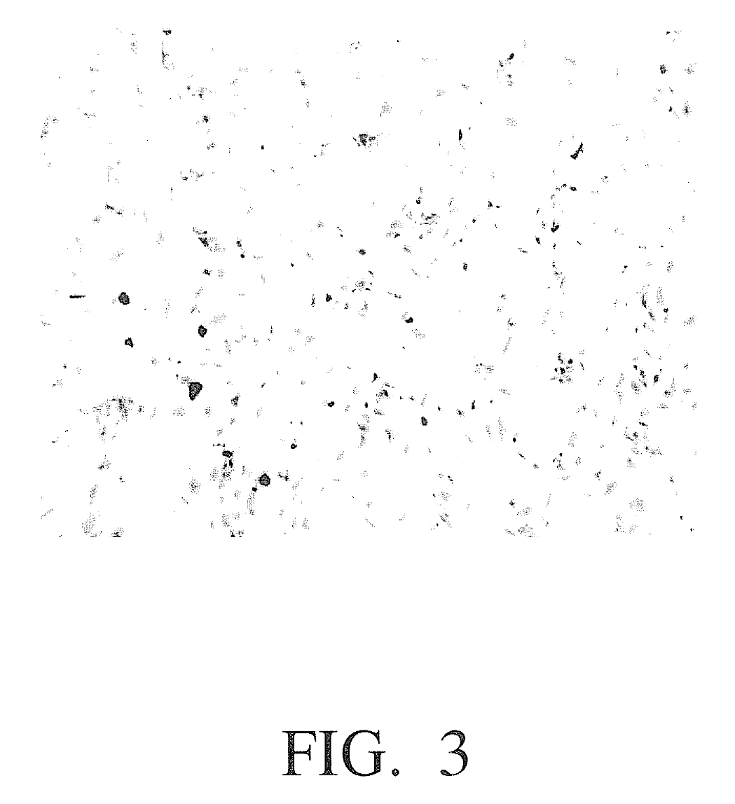

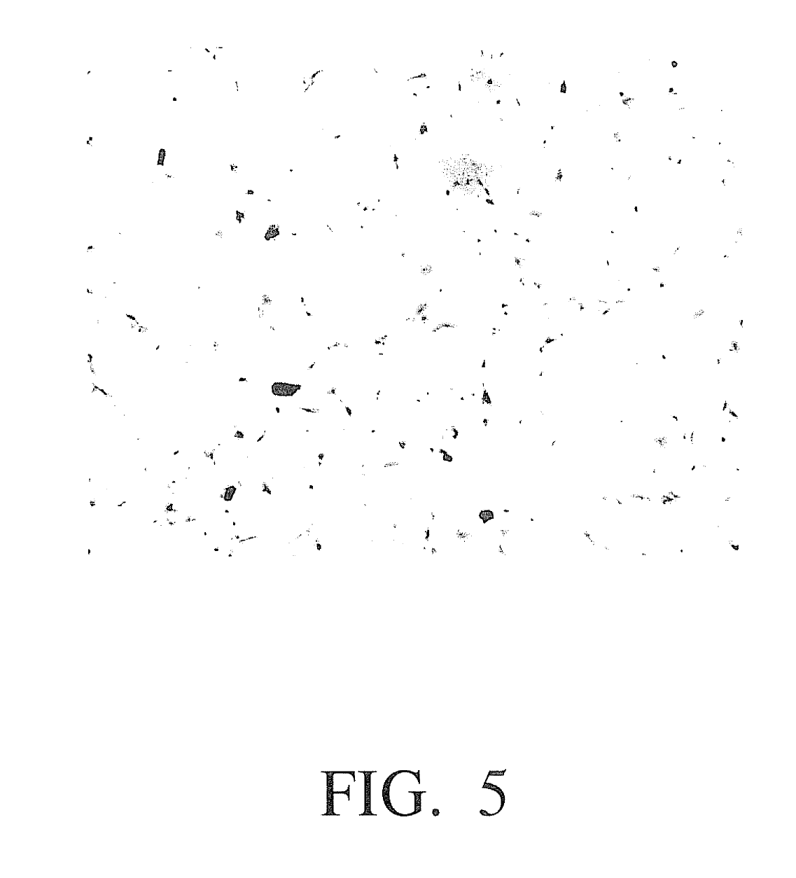

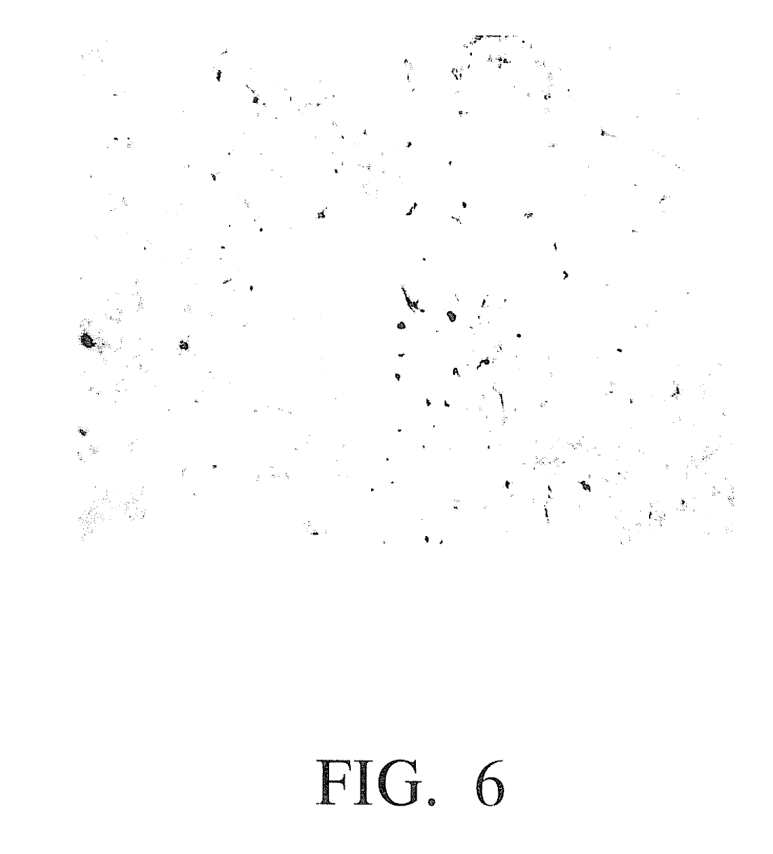

As examples, observation images of the HUV-EC-C cells cultured using the cell culture carriers of the Example 1 and the Comparative Examples 1 to 3 are shown in FIGS. 3 to 6, respectively.

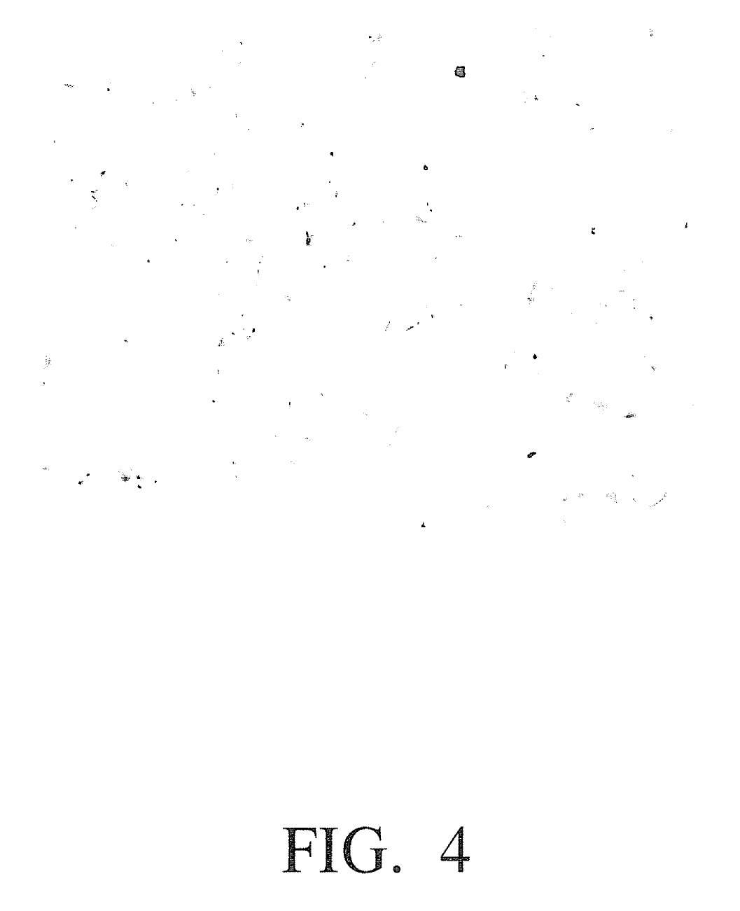

It is to be noted that in FIGS. 3 to 6, HUV-EC-C cells are darker in color. As can be seen from FIG. 3, the HUV-EC-C cells cultured using the cell culture carriers of the Example 1 were adsorbed to the cell culture carriers and the number of grown cells was larger as compared to the cases shown in FIGS. 4 to 6. On the other hand, as can be seen from FIGS. 4 to 6, the numbers of HUV-EC-C cells cultured using the cell culture carriers of the Comparative Examples 1 to 3, respectively were smaller as compared to the case shown in FIG. 3.

Next, the HUV-EC-C cells cultured using the cell culture carriers of each of the Examples 1 to 13 and the Comparative Examples 1 to 3 were observed with a microscope to count the number of cells per unit area of the surface of each of the cell culture carriers.

It is to be noted that the number of cells was determined by averaging the number of cells cultured using the five cell culture carriers, and was expressed in terms of a relative ratio of the thus obtained average value with respect to the average value of the Comparative Example 3 (the relative ratio of the Comparative Example 3 was defined as 1.0). The results are shown in Table 1.

TABLE 1

| | | | | | | | | | | | | | | Evaluation Result | |
|---|---|---|---|---|---|---|---|---|---|---|---|---|---|---|---|
| | | | | | | | | | | | | | | Obser- | Number |
| | | | | | Manufacturing Conditions | | | | | | | | | vation | of |
| | | | | | | First Treatment Liquid | | | | Second Treatment Liquid | | | | Image | Cells |
| | Composition of Cell Culture Carrier | | | | | Protein | | | | Collagen | | | | by | (Rel- |
| | Base | | Collagen | | | Conc. | Temp. | Time | | Conc. | Temp. | Time | | Micro- | ative |
| | Material | Protein | Type | Source | Method | [mg/mL] | [° C.] | [min] | pH | [mg/mL] | [° C.] | [min] | pH | scope | Ratio) |
| Ex. 1 | HAP | FN | I | swine | a | 5 | 37 | 30 | 7.4 | 100 | 37 | 30 | 7.4 | | 1.98 |
| Ex. 2 | TCP | FN | I | swine | a | 5 | 37 | 30 | 7.4 | 100 | 37 | 30 | 7.4 | — | 1.87 |
| Ex. 3 | HAP + TCP | FN | I | swine | a | 5 | 37 | 30 | 7.4 | 100 | 37 | 30 | 7.4 | — | 1.91 |
| Ex. 4 | matrix: PS | FN | I | swine | a | 5 | 37 | 30 | 7.4 | 100 | 37 | 30 | 7.4 | — | 1.90 |
| | surface | | | | | | | | | | | | | | |
| | layer: HAP | | | | | | | | | | | | | | |
| Ex. 5 | HAP | INT | I | swine | a | 5 | 37 | 30 | 7.4 | 100 | 37 | 30 | 7.4 | — | 2.01 |
| Ex. 6 | HAP | FN + | I | swine | a | 5 | 37 | 30 | 7.4 | 100 | 37 | 30 | 7.4 | — | 2.05 |
| | | INT | | | | | | | | | | | | | |
| Ex. 7 | HAP | FN | II | swine | a | 5 | 37 | 30 | 7.4 | 100 | 37 | 30 | 7.4 | — | 1.71 |
| Ex. 8 | HAP | FN | I | salmon | a | 5 | 37 | 30 | 7.4 | 100 | 37 | 30 | 7.4 | — | 1.66 |
| Ex. 9 | HAP | FN | I | swine | a | 0.1 | 37 | 30 | 7.4 | 100 | 37 | 30 | 7.4 | — | 1.67 |
| Ex. 10 | HAP | FN | I | swine | a | 100 | 37 | 30 | 7.4 | 100 | 37 | 30 | 7.4 | — | 1.88 |
| Ex. 11 | HAP | FN | I | swine | a | 5 | 37 | 30 | 7.4 | 1 | 37 | 30 | 7.4 | — | 1.70 |
| Ex. 12 | HAP | FN | I | swine | a | 5 | 37 | 30 | 7.4 | 1,000 | 37 | 30 | 7.4 | — | 1.89 |
| Ex. 13 | HAP | FN | I | swine | b | (5) | (37) | (30) | (7.4) | (100) | (37) | (30) | (7.4) | — | 1.80 |
| Comp. | HAP | — | I | swine | — | — | — | — | — | 100 | 37 | 30 | 7.4 | | 0.78 |
| Ex. 1 | | | | | | | | | | | | | | | |
| Comp. | HAP | FN | — | — | — | 5 | 37 | 30 | 7.4 | — | — | — | — | | 1.39 |
| Ex. 2 | | | | | | | | | | | | | | | |
| Comp. | HAP | — | — | — | — | — | — | — | — | — | — | — | — | | 1.00 |
| Ex. 3 | | | | | | | | | | | | | | | |

HAP: hydroxyapatile
TCP: tricalcium phosphate
PS: polystyrene resin
FN: fibronectin
INT: integrin
a: first treatment liquid (containing protein) + second treatment liquid (containing collagen)
b: treatment liquid (containing protein and collagen)
In columns of manufacturing conditions. values within parentheses are data of the treatment liquid b.

As can be seen from Table 1, the number of cells cultured using the cell culture carriers of the Example 1 was about 1.4 to 2.5 times that of each of the Comparative Examples 1 to 3. Also, the number of cells cultured using the cell culture carriers of each of the Examples 2 to 13 was about 1.2 to 2.6 times that of each of the Comparative Examples 1 to 3.

From the result, it can be considered that the collagen-coated carriers of each of the Examples 1 to 13 firmly adsorbed HUV-EC-C cells so that the cells did not come off from the collagen-coated carriers, thereby promoting the growth of the cells.

On the other hand, as described above, the cell culture carrier of the Comparative Example 1 does not have a protein having a high affinity for collagen, the cell culture carrier of the Comparative Examples 2 does not have collagen, and the cell culture carrier of the Comparative Example 3 is an untreated carrier of hydroxyapatite. From the fact, it can be considered that the HUV-EC-C cells easily came off from the cell culture carriers of the Comparative Examples 1 to 3 due to weak adsorption of the HUV-EC-C cells to the cell culture carriers so that the cells did not sufficiently grow.

3. Bone Filling Material (Artificial Bone) Implantation Test

First, bone filling materials were manufactured in the same manner as in the Examples 1 to 13 and the Comparative Examples 1 to 3, respectively except that the base material was changed to a pellet having a diameter of 5 mm and a thickness (length) of 10 mm.

Next, Japanese white domestic rabbits were prepared, and a hole having a diameter of 5.5 mm and a depth of 10.5 mm was drilled in the condyle of the femur of each of the rabbits. The holes of these rabbits were filled with the bone filling materials of the Examples 1 to 13 and the Comparative Examples 1 to 3, respectively.

After a lapse of six weeks, the rabbits were killed. The site filled with the bone filling material in the condyle of the femur of each of the rabbits was stained by HE staining, and was then observed with a microscope.

As a result, in each of the sites filled with the bone filling materials of the Examples 1 to 13, respectively, regenerated bone tissue and the bone filling material were being fused together in spite of a relatively short period of implantation (6 weeks). This result indicates that the bone filling materials of the Examples 1 to 13 fulfilled their functions satisfactorily.

On the other hand, in each of the sites filled with the bone filling materials of the Comparative Examples 1 to 3, respectively, a boundary between the bone filling material and new bone tissue was clearly observed. This result indicates that new bone tissue and the bone filling material were poorly fused together.

Effect of the Invention

According to the present invention, it is possible to efficiently and reliably obtain a collagen-coated carrier that has excellent cell adhesion properties and that allows excellent cell growth thereon.

In a case where the collagen-coated carrier according to the present invention is used as a cell culture carrier, cells to be cultured grow more efficiently and reliably.

Further, in a case where the collagen-coated carrier according to the present invention is used as a material for filling a bone defect site, the carrier serves as a scaffold that allows new bone tissue (osteoblasts) to more efficiently grow thereon. In this case, the collagen-coated carrier and grown osteoblasts repair and regenerate the bone defect site faster.

Finally, it is also to be understood that the present disclosure relates to subject matter contained in Japanese Patent Application No. 2005-152778 (filed on May 25, 2005) which is expressly incorporated herein by reference in its entirety.

What is claimed is:

1. A collagen-coated carrier, comprising a carrier having a surface, in which at least part of the surface of the carrier being composed of a calcium phosphate-based compound, wherein the part of the surface of the carrier is coated with collagen via a protein having a high affinity for the collagen.

2. The collagen-coated carrier as claimed in claim 1, wherein the protein has a collagen receptor.

3. The collagen-coated carrier as claimed in claim 2, wherein the protein contains at least one of fibronectin and integrin as a main ingredient.

4. The collagen-coated carrier as claimed in claim 1, wherein the collagen contains type I collagen as a main ingredient.

5. The collagen-coated carrier as claimed in claim 1, wherein the collagen is derived from a land animal.

6. The collagen-coated carrier as claimed in claim 1, wherein at least part of the collagen is denatured.

7. The collagen-coated carrier as claimed in claim 1, wherein the collagen can be dissolved in a solvent having a pH of 6.0 to 8.0 at a ratio of 100 μg or more per milliliter of the solvent.

8. The collagen-coated carrier as claimed in claim 1, wherein the carrier is obtained by coating the surface of a matrix with the calcium phosphate-based compound.

9. The collagen-coated carrier as claimed in claim 1, wherein the carrier has a granular, pellet, block, or sheet shape.

10. The collagen-coated carrier as claimed in claim 1, wherein the calcium phosphate-based compound contains at least one of tricalcium phosphate and hydroxyapatite as a main ingredient.

11. The collagen-coated carrier as claimed in claim 1, which is intended for use in cell culture.

12. The collagen-coated carrier as claimed in claim 1, which is intended for use in filling a bone defect site.

13. A method for manufacturing a collagen-coated carrier, comprising the steps of:
preparing a carrier having a surface, in which at least part of the surface of the carrier being composed of a calcium phosphate-based compound; and
bringing the carrier into contact with collagen and a protein having a high affinity for the collagen to coat the part of the surface of the carrier with the collagen via the protein.

14. A method for manufacturing a collagen-coated carrier, comprising the steps of:
preparing a carrier having a surface, in which at least part of the surface of the carrier being composed of a calcium phosphate-based compound;
bringing the carrier into contact with a first treatment liquid containing a protein having a high affinity for collagen to allow the protein to adhere to the part of the surface of the carrier; and
bringing the carrier into contact with a second treatment liquid containing the collagen to coat the part of the surface of the carrier with the collagen via the protein.

15. The method for manufacturing a collagen-coated carrier as claimed in claim 14, wherein the protein concentration in the first treatment liquid is 01 to 100 μg/mL.

16. The method for manufacturing a collagen-coated carrier as claimed in claim 14, wherein the temperature of the first treatment liquid is 4 to 39° C.

17. The method for manufacturing a collagen-coated carrier as claimed in claim 14, wherein the time during which the carrier is kept in contact with the first treatment liquid is 10 minutes to 10 hours.

18. The method for manufacturing a collagen-coated carrier as claimed in claim 14, wherein the pH of the first treatment liquid is 6.0 to 8.0.

19. The method for manufacturing a collagen-coated carrier as claimed in claim 14, wherein the collagen concentration in the second treatment liquid is 1 to 1,000 μg/mL.

20. The method for manufacturing a collagen-coated carrier as claimed in claim 14, wherein the temperature of the second treatment liquid is 4 to 39° C.

21. The method for manufacturing a collagen-coated carrier as claimed in claim 14, wherein the time during which the carrier is kept in contact with the second treatment liquid is 10 minutes to 10 hours.

22. The method for manufacturing a collagen-coated carrier as claimed in claim 14, wherein the pH of the second treatment liquid is 6.0 to 8.0.
